(19) United States
(12) Patent Application Publication
(10) Pub. No.: US 2003/0165502 A1
(43) Pub. Date: Sep. 4, 2003

(54) Single-chain antibodies against human insulin-like growth factor I receptor: expression, purification, and effect on tumor growth

(75) Inventor: Yoko Fujita-Yamaguchi, Duarte, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope

(21) Appl. No.: 10/134,519

(22) Filed: Apr. 30, 2002

Related U.S. Application Data
(63) Continuation of application No. 09/609,776, filed on Jul. 3, 2000, now abandoned.
(60) Provisional application No. 60/211,187, filed on Jun. 13, 2000.

(51) Int. Cl. A61K 39/395; C07K 16/22

(52) U.S. Cl. 424/145.1; 530/388.25

(57) ABSTRACT

A method of inhibiting the growth of hormone dependent tumor cells in a mammal comprises administering to said mammal an insulin-like growth factor receptor (IGF-IR) recombinant antibody, wherein said antibody can be a single-chain recombinant antibody, which can be humanized, capable of blocking agonist interaction with the IGF-IR.

This application is a continuation of U.S. application Ser. No. 09/609,776, filed Jul. 3, 2000, which claims priority from provisional application No. 60/211,187, filed Jun. 13, 2000.

### FIELD OF THE INVENTION

The present invention is in the field of methods for treatment of hormone dependent cancers.

### BACKGROUND OF THE INVENTION

Insulin and Insulin-like Growth Factors stimulate the growth of human breast cancer cells in vitro. The Insulin-like Growth Factors I (IGF-I) and II (IGF-II) interact with cell surface receptors eliciting their cellular response. The IGF-I receptor (IGF-IR) is the cell surface receptor for IGF-I having high binding affinity for this growth factor. However, IGF-IR is also thought to have a high binding affinity for IGF-II. Interaction of either of these two growth factors to the IGF-IR elicits intracellular responses through protein tyrosine phosphorylations, which can be blocked through the inhibition of the interaction of either IGF-I or IGF-II to the receptor.

These intracellular responses of IGF-IR signaling are implicated in the inducement of cell growth, proliferation and anti-apoptosis. It has been shown that the IGF-IR can not only induce normal cell growth but also induces tumor cell growth in both breast cancer and prostate cancer. In addition, the anti-apoptotic activity of IGF-IR protects cancerous tumor cells from chemotherapeutic treatments in breast cancers.

Therefore, a need exists for a method of inhibiting IGF-IR in order to inhibit tumor cell growth and increase sensitivity to chemotherapeutic agents. The activity of the IGF-IR can be inhibited by various methods. One of these methods comprises inhibiting the activation of the IGF-IR by preventing binding of agonist such as IGF-I or IGF-II. This can be achieved by blocking the IGF-IR binding site with antagonists.

Antibodies can be effective antagonists in inhibiting the interaction of the IGF-IR with IGF-I or IGF-II. αIR-3 (Arteaga, C. L. and Osborne, C. K.; Cancer Research 49, 6237-6241, 1989) is an antibody with high affinity for the IGF-IR and has been found to inhibit the interaction of IGF-I with the IGF-IR. In in vitro experimentation this murine antibody has been found to inhibit the growth of various tumor cells from breast cancer cell lines. In various tumor cells (MCF-7, MDA-231, ZR75-1, and HS578T) this αIR-3 could inhibit the IGF-I mediated DNA synthesis in vitro. However, in estrogen dependent tumor cells, such as MCF-7, ZR75-1 and T47D, the inhibition with αIR-3 of the IGF-IR in vivo failed to block estrogen stimulated DNA synthesis or proliferation. In contrast, in T61 tumor cells the αIR-3 antibody could inhibit tumor cell growth in vivo when used in combination with down-regulation of IGF-II synthesis by simultaneous treatment with estradiol and tamoxifen. It appears that αIR-3 is a better antagonist for IGF-I blockage compared to its ability to inhibit interaction of IGF-II with IGF-IR.

Another murine antibody against the α-subunit of IGF-IR, 1H7 (Li S. et al; Biochemical and Biophysical Research Communications, 196, 92-98, 1993), has shown good results in inhibiting the activation of IGF-IR. In in vitro experimentation with NIH3T3 cells over-expressing human IGF-IR the 1H7 antibody inhibits basal, IGF-I or IGF-II stimulated DNA synthesis. A second antibody raised against the IGF-IR α-subunit, 2C8, however, is unable to block IGF-IR activation by either IGF-I or IGF-II while having binding affinities for the receptor.

While these two murine antibodies, αIR-3 and 1H7, have shown results in inactivation of the IGF-IR in vitro, their ability to inhibit estrogen dependent tumor cell growth in vivo is limited. Furthermore, the monoclonal murine antibodies have their obvious disadvantages in their use for human treatment or other mammals. In addition, their relative complexity limits the ability to manipulate the antibodies to optimize their use in the treatment of mammalian hormone dependent cancers. Accordingly, improvements are sought.

### SUMMARY OF THE INVENTION

In accordance with the present invention, a method of inhibiting the growth of hormone dependent tumor cells in a mammal comprises administering to said mammal an anti insulin-like growth factor I receptor (IGF-IR) recombinant antibody. In a preferred embodiment, the method comprises administering a single chain antibody (scFv). In a further preferred embodiment the method comprises administering a chimeric single chain antibody in which a constant domain has been linked to the single chain antibody.

There also is provided a novel IGF-IR antagonist comprising a recombinant single-chain antibody which blocks agonist interaction with the IGF-IR. In one embodiment of the invention, the antibody is in the form of a novel chimeric single-chain antibody against IGF-IR.

### BRIEF DESCRIPTION OF THE FIGURES

FIG. 1. This figure is a schematic representation of soluble forms of anti (human insulin-like growth factor I receptor) single-chain antibodies (αIGF-IR scFvs). A is a single-chain antibody (αIGF-IR scFv). B is a chimeric αIGF-IR scFv-Fc.

FIG. 2. This figure illustrates the effects of αIGF-IR scFv-Fc and mAb on ¹²⁵I-IGF-II (A) and ¹²⁵I-IGF-I (B) binding to purified human IGF-1 receptor. The binding activity is calculated as the percentage IGF-binding in the absence of antibodies, and expressed as average±SD of four independent experiments for A or seven independent experiments for B, except that two control experiments with 2C8 mAb were performed for B. Antibodies used are αIGF-IR scFv-Fc (▾), 1H7 (▪), and control 2C8 (▴).

FIG. 3. This figure illustrates the effects of αIGF-IR scFv-Fc and 1H7 on cell growth. NIH3T3 cells over-expressing IGF-IR were cultured in the absence (▪), or presence of 10 nM (▴) , 100 nM (▾) or 1000 nM (♦) 1H7 (A) or αIGF-IR scFv-Fc (B).

FIG. 4. This figure illustrates the effects of MCF-7 tumor cell growth in nude mice in the absence (▪) or presence (▴) of αIGF-IR scFv-Fc.

FIG. 5. This figure illustrates the effects of αIGF-IR scFv-Fc on MCF-7 tumor cell growth in vivo in the presence or absence of the anti-neoplastic agent Doxorubicin. On day 0 MCF-7 cells were implanted in the mouse followed by a treatment of PBS (control) (▪), αIGF-IR scFv-Fc (▴), Doxorubicin (▾) or αIGF-IR scFv-Fc+Doxorubicin (♦) beginning at day 4.

FIG. 6. This figure illustrates the effects of αIGF-IR scFv-Fc on T61 tumor cell growth in vivo in the presence or absence of the estrogen antagonist Tamoxifen (TAM). On day 36 following implantation of T61 tumor cells the mice were treated with PBS (control) (●), αIGF-IR scFv-Fc (Δ), tamoxifen (▪) or αIGF-IR scFv-Fc+tamoxifen (*).

### DETAILED DESCRIPTION OF THE INVENTION

The present invention provides a method of inhibiting hormone dependent tumor growth by blocking the activation of the Insulin-like Growth Factor I receptor (IGF-IR). This blockage can be accomplished by exposing hormone dependent tumor cells to an antagonist of IGF-IR. Inhibition of IGF-IR can lead to a decrease in cell growth and can also render the hormone dependent tumor cells more susceptible to therapeutic agents. Alternatively, the antagonist interaction with IGF-IR, inhibiting the activation of the receptor, can lead to apoptosis of the hormone dependent tumor cells.

Therefore, the invention provides a method of treatment of mammals suffering from hormone dependent tumor cell growth by administering to the mammal an anti-IGF-IR recombinant antibody. Preferably the invention provides for a treatment of mammals suffering from estrogen dependent cancer, such as breast cancer. In addition, the treatment comprises administering to said mammal an anti-IGF-IR recombinant antibody in combination with one or more therapeutic agents, such as tamoxifen which are effective in reducing the growth of hormone-dependent tumors.

In a preferred embodiment of the invention the anti-IGF-IR antibody is a single chain recombinant antibody, such as the 1H7 based αIGF-IR scFv or αIGF-IR scFv-Fc antibodies. The single chain antibodies are advantageous because of the relative ease in their expression, purification and manipulation. The expression of such antibodies in expression systems makes them more susceptible to large scale production and purification. In addition, manipulation of such single chain antibodies may consist of altering such antibodies to covalently attach other therapeutic agents. Such agents can, for example, include toxins, enzymes, or radionucleotides. The recombinant single chain antibody conjugated with such agents can block IGF-IR induced tumor cell growth and target such agents to said tumor cells which have been made more susceptible to apoptosis by the inhibition of IGF-IR.

The single chain antibody comprises at least an Fv domain capable of blocking IGF-IR interaction with IGF-I or IGF-II. The IGF-IR scFv comprises both the antigen binding region of a light chain variable domain, V_{L}, and the antigen binding region of a heavy chain variable domain, V_{H}, coupled by a short linker peptide. In a preferred embodiment, the V_{L} domain and the V_{H} domain are derived from the 1H7 antibody against the α-subunit of IGF-IR. The IGF-IR scFv can be tagged with a short peptide such as the FLAG epitope to facilitate purification of the soluble IGF-IR scFv from the medium of the expression system. The DNA coding for the V_{L} and V_{H} domains are obtainable by sequencing said domains from a parental antibody, in a preferred embodiment said parental antibody being 1H7. A recombinant DNA then can be constructed comprising, in order, coding sequences for the N-terminal signal peptide, the antigen binding region of the V_{L} domain, a linker peptide, the antigen binding region of the V_{H} domain and a C-terminal tag peptide for purification and identification. Said genetically engineered antibody can be expressed in myeloma or bacterial cell expression systems. The monovalent recombinant single chain antibody IGF-IR scFv can be purified from the medium of said expression system by conventional protein purification methods, such as, for example, affinity chromatography.

The linker peptide is chosen based upon known structural and conformational information of peptide segments and is selected so that it will not interfere with the tertiary structure of the single chain antibody and its uses. Typically, a linker of between about 6 and 50 amino acids is preferred for ease and economics of preparation.

In one embodiment of the invention the soluble IGF-IR scFv is a chimeric antibody which further comprise an Fc domain. In this embodiment, the recombinant DNA will comprise the coding sequence of IGF-IR scFv minus the C-terminal tag peptide, coupled to a coding sequence for an Fc domain. Desirably, the Fc domain comprises the C_{H2} and C_{H3} regions of an antibody heavy chain constant domain. The recombinant DNA can be expressed in a myeloma or bacterial expression system in accordance with conventional techniques and said single-chain antibody IGF-IR scFv-Fc can be purified using conventional protein purification methods. The IGF-IR scFv-Fc exists preferably in its divalent form. The IGF-IR scFv-Fc can comprise a humanized form of the IGF-IR scFv, such as, for example, by using a coding sequence of a human Fc domain when constructing the recombinant DNA. Said single chain antibodies (IGF-IR scFv or IGF-IR scFv-Fc) subsequently can be modified, if desired, and attached to other therapeutic agents.

To treat mammals suffering from hormone dependent cancer, preferably from estrogen dependent breast cancer, the recombinant single-chain antibodies (IGF-IR scFv or IGF-IR scFv-Fc) can be administered in a pharmaceutically acceptable composition as the sole therapeutic or in combination with one or more other therapeutic agents, such as tamoxifen, which are effective in reducing hormone-dependent tumor cell growth. The tamoxifen or other therapeutic agent can be administered in accordance with conventional therapeutic methods, such as parenteral or subcutaneous administration. Administration of said recombinant single chain antibodies can be used as a method of inhibiting tumor cell growth in vivo or to induce susceptibility of said tumor cells to therapeutic agents.

In light of the preceding description, one skilled in the art can use the present invention to its fullest extent. The following examples, therefore, are to be construed as illustrative only and not limiting in relation to the remainder of the disclosure.

### EXAMPLE 1

Cloning of 1H7 Variable Domains by RT-PCR.

Heavy and light chains of mouse monoclonal antibody 1H7 (Li, S. et al; Biochemical and Biophysical Research Communications, 196, 92-98, 1993) were separated by sodium dodecyl sulfate/polyacrylamide gel electrophoresis (SDS-PAGE; 12.5% polyacrylamide gel), under reducing conditions, blotted onto a polyvinylidene difluoride membrane, and subjected to N-terminal amino acid sequence determination by Edman degradation. Degenerate oligonucleotides, used as upstream primers, were synthesized on the N-terminal sequences of the heavy and light chains of 1H7 while the constant region oligonucleotides for the downstream primers were designed and synthesized according to the published nucleotide sequences. Primers (Table 1) containing the EcoRI site were used to amplify the heavy- and light-chain variable regions (V_{H} and V_{L}, respectively) from 1H7 poly(A) rich mRNA by reverse transcriptase polymerase chain reaction (RT-PCR). PCR products were ligated into the EcoRI site of pBleuscriptII SK. Escheria coli XL1-Blue was transformed with the vectors encoding PCR-generated V_{H} and V_{L} sequences.

The N-terminal amino acid sequences of the heavy- and light-chains of 1H7 were determined to be EVKVVESGGGLVKPG (SEQ ID: NO 1) and DIVMTQSHKFMSTSV (SEQ ID: NO 2) respectively.

TABLE 1

Primers for PCR amplification of variable
regions of heavy and light chains of 1H7.
Light-chain primers

| | |
|---|---|
| Amino Acid | 1 2 3 4 5 6 |
| | Asp Ile Val Met Thr Gln |
| 5′ End primer gggaattc | G A C A T T G T G A T G A C C C A A 3′ |
| | T C C A G |
| | T |
| C-region amino acid | Ser Ile Phe Pro Pro Ser |
| C-region primer | 5′ T C C A T C T T C C C A C C A T C C g a a t t c c g 3′ |

Heavy-chain primers

| | |
|---|---|
| Amino Acid | 1 2 3 4 5 6 |
| | Glu Val Lys Val Val Glu |
| 5′ End primer gggaattc | G A A G T A A A A G T A G T A G A A 3′ |
| | G C G C C G |
| | G G G |
| C-region amino acid | Val Tyr Pro Leu Ala Pro |
| C-region primer | 5′ G T C T A T C C A C T G G C C C C T gaattccg 3′ |

### EXAMPLE 2

Design of αIGF-IR Antibodies.

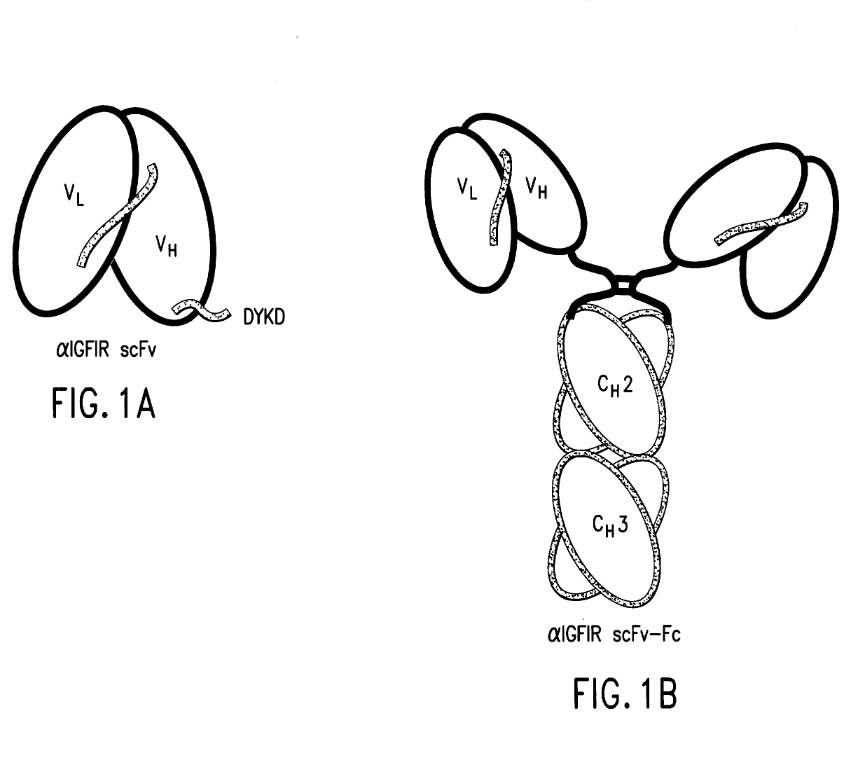

Two soluble forms of 1H7-based αIGF-IR antibodies, scFv and scFv-Fc, were designed as schematically presented in FIG. 1. ScFv is a monovalent antibody and has an expected Mᵣ of 27 kDa. ScFv-Fc is a divalent antibody that contains the human IgG1 Fc domain and has an expected Mᵣ of 120 kDa.

The gene encoding the αIGF-IR scFv was constructed using the N-terminal signal peptide derived from the mT84.66 light chain, V_{L} DNA, an oligonucleotide encoding the linker peptide (GGGGSGGGS)₂, V_{H} DNA, and a C-terminal tag (including DYKD), and assembled using splice-overlap extension PCR. The αIGF-IR scFv construct was cloned into pcDNA3 (Invitrogen, San Diego, Calif.), containing the cytomegalovirus promoter and neo^{r} selection marker (pcDNA/αIGF-IR scFv).

To construct the gene encoding αIGF-IR scFv-Fc, a SalI fragment containing the human IgG1 Fc (cDNA clone from Dr. J. Schlom, Laboratory of Tumor Immunology and Biology, division of Cancer Biology and Diagnosis, NCI, Bethesda, Md.) was inserted into the unique XhoI site of pcDNA/αIGF-IR scFv. The HindIII fragment encoding αIGF-IR scFv-Fc, isolated from the pcDNA/αIGF-IR scFv-Fc plasmid, was inserted into the HindIII site of pEE12-1. This plasmid encodes a glutamine synthase gene that provides a selection system for myeloma NS0 cells in L-glutamine-deficient selection medium.

### EXAMPLE 3

Cell Culture, Transfection and Purification of αIGF-IR scfv or αIGF-IR scFv-Fc.

Murine myeloma Sp2/0 cells were transfected with pcDNA/αIGF-IR scFv by electroporation, and incubated at 37° C. for 3 days in a humidified 5% CO₂ atmosphere. On day 4, cells were collected, counted and placed in 24-well plates (10⁵ cells/well) in regular medium containing 400 μg/ml G418. Murine myeloma NS0 cells were grown in selective medium consisting of L-glutamine-free Celltech DME (JRH Biosciences, Lenexa, Kans.), dialyzed fetal calf serum (Gibco/BRL, Gaithersburg, Md.), and glutaminase synthase supplement (JRH Biosciences, Lenexa, Kans.). Murine myeloma NS0 cells were stably transfected with pEE12-1/αIGF-IR scFv-Fc by electroporation and transferred to non-selective culture medium in a 96-well plate (50 μl/well), and incubated overnight. The next day 150 μl of selection medium was added to each well, and the cells were incubated for three weeks until discrete surviving colonies appeared.

To purify αIGF-IR scFv by affinity chromatography, 150 ml of conditioned medium (CM), collected from Sp2/0 cells, were applied to 6 ml αFLAG M2 mAb (Eastman Kodak Co., Rochester, N.Y.) conjugated to Sepharose 4B (0.2 mg/ml gel), and αIGF-IR scFv-Fc was eluted from the column with FLAG peptide. Eluates were concentrated and dialyzed, using an Ultrafree-4 spin column (Millipore, Bedford, Mass.). Based on the recovery of approximately 4 μg of αIGF-IR scFv protein from purifying 150 ml CM, the level of αIGF-IR scFv expression was estimated to be approximately 20 ng/ml CM.

To purify αIGF-IR scFv-Fc approximately 40 ml cell culture supernatants collected from αIGF-IR scFv-Fc expressing NS0 transfectants were adjusted to pH 8.0 by adding 1/20 volume 1.0M TRIS (pH 8.0), and passed through a protein-A-Sepharose CL 4B column. αIGF-IR scFv-Fc was eluted from the column with 100 mM glycine buffer pH 3.0, collected in 1.5 ml conical tubes containing 1/10 volume 1M TRIS (pH 8.0). The estimated expression level of αIGF-IR scFv-Fc in this expression system ranged between 45 μg/ml and 85 μg/ml.

### EXAMPLE 4

Inhibition of Agonist Binding to Purified IGF-IR by 1H7 and αIGF-IR scFv-Fc.

The affinity constants of 1H7 (10⁹ M⁻¹) and αIGF-IR scFv-Fc (10⁸ M⁻¹) for IGF-IR were determined using a BIAcore instrument (BIAcore Inc., Piscataway, N.J.). Analytes, at various concentrations, were passed over IGF-IR-immobilized chips (0.3 μg/chip) at a flow rate of 5 μl/min.

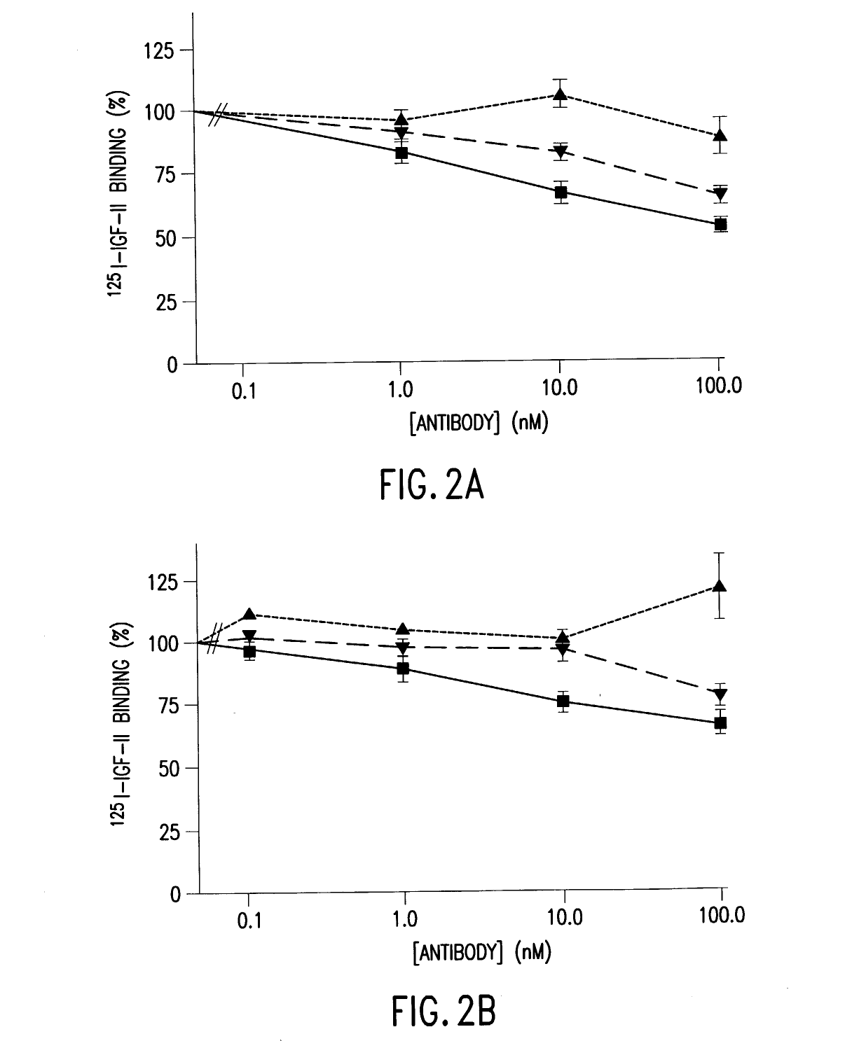

The in vitro potency of inhibition of purified IGF-IR by αIGF-IR scFv-Fc for both IGF-I and IGF-II binding is seen in FIG. 2.

### EXAMPLE 5

Effect of αIGF-IR scFv-Fc on Cell Growth.

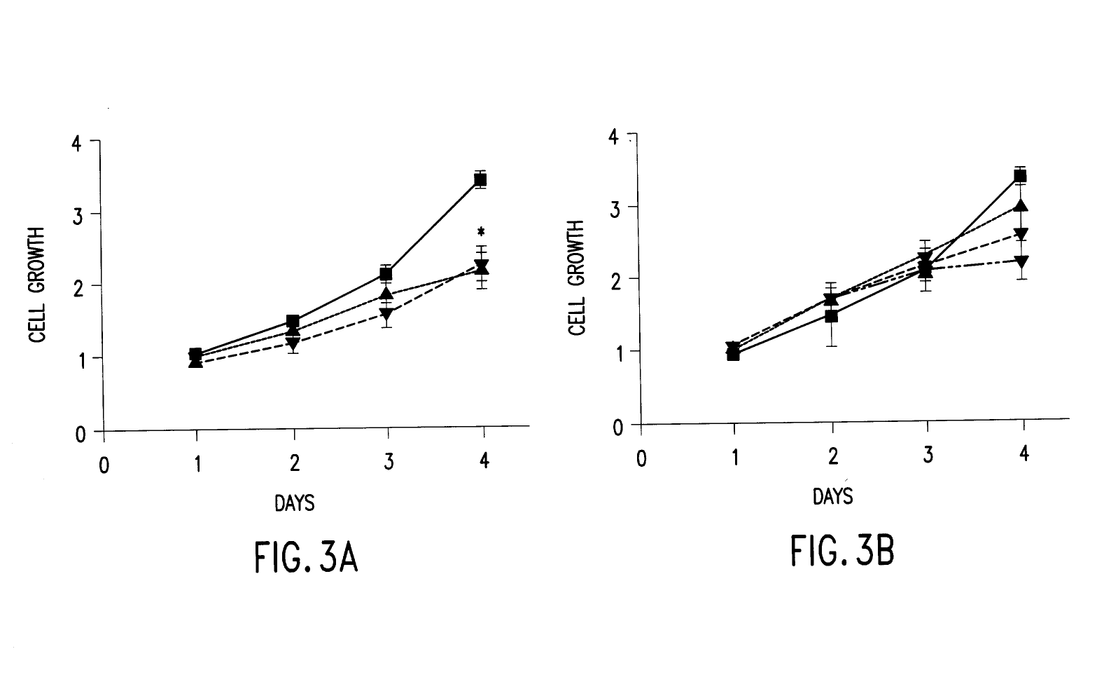

Using the MTT method the effect of extracellular addition of αIGF-IR scFv-Fc or 1H7 on cell growth was determined on NIH3T3 cells over expressing IGF-IR. Cell growth was significantly inhibited after four days of treatment with 10 nM or 100 nM 1H7 mAb, see FIG. 3. Also, after 4 days of treatment with αIGF-IR scFv-Fc cell growth appeared to be inhibited in a dose dependent manner as is shown in FIG. 3.

### EXAMPLE 6

Effect of αIGF-IR scFv-Fc on Tumor Growth in Vivo.

The human breast MCF-7 cell line was obtained from American Type Culture Collection (Rockville, Md.). MCF-7 cells were cultured in Dulbecco's modified Eagle's medium supplemented with 5% fetal calf serum. Female athymic mice (BALB/C nude, Charles River Facility for NCI, Frederick, Md.), 4 weeks old, that had received 0.25 mg 17β-estradiol pellet one week previously were inoculated in the flank with 10⁷ MCF-7 cells (day 0). On day 3, intraperitoneal or subcutaneous injections near the tumor sites of αIGF-IR scFv-Fc into each of three mice (500 μg/0.1 ml phosphate buffered saline, PBS/mouse, twice a week) was started, and continued for two weeks.

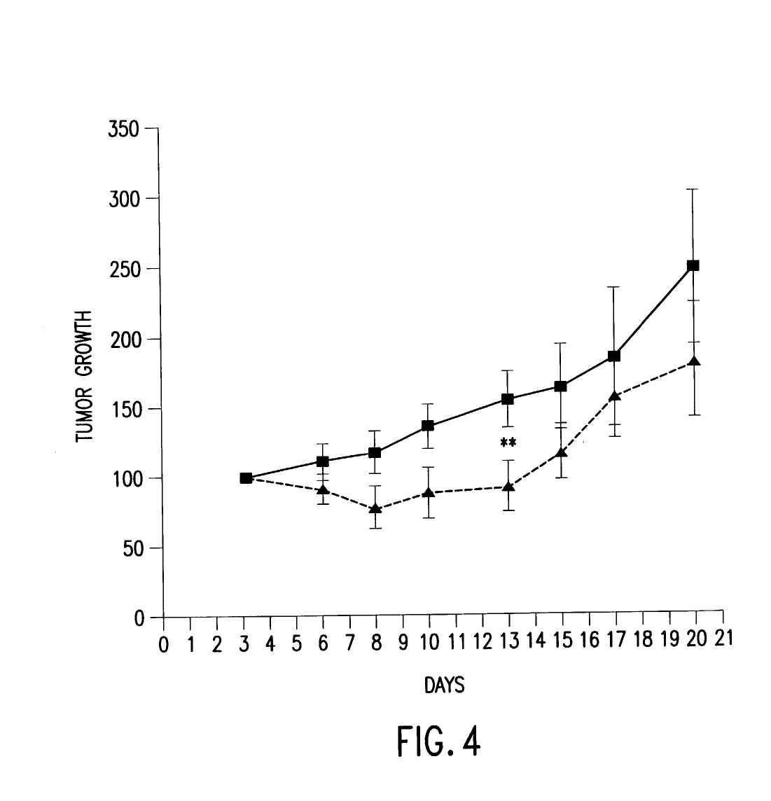

The recombinant single chain antibody αIGF-IR scFv-Fc inhibits MCF-7 tumor cell growth in athymic mice. As shown in FIG. 4, inhibition of tumor cell growth is significant, although, the results for individual mice varied. In several mice MCF-7 tumor cell growth was completely suppressed from day 3 to day 17, and in one mouse the tumor disappeared.

### EXAMPLE 7

Effect of αIGF-IR scFv-Fc in Combination With Anti-neoplastic Agent Doxorubicin (DOX) on Tumor Growth in Vivo.

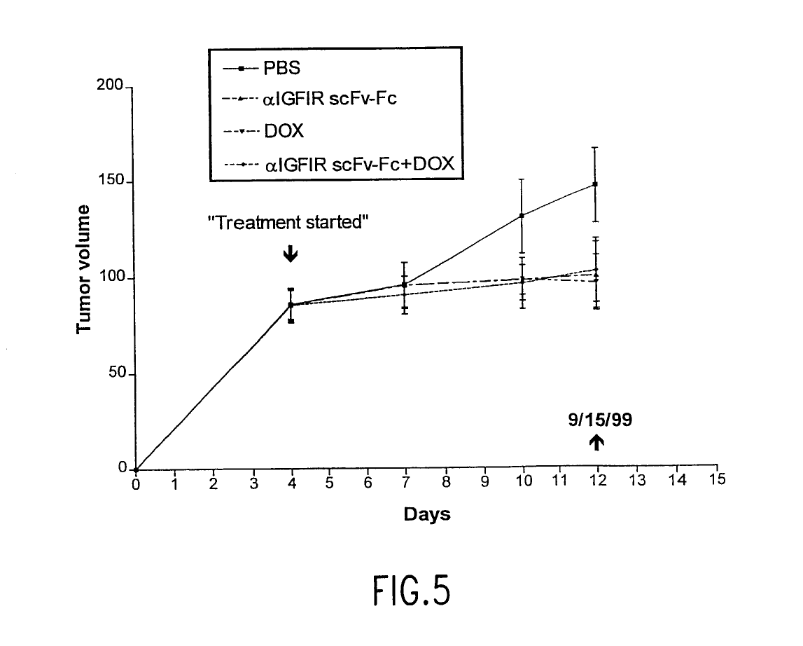

In combination with Doxorubicin (DOX), αIGF-IR scFv-Fc inhibits tumor cell growth, as shown in FIG. 5. Female athymic mice were treated similarly as described above in example 6, with the exception that the pellet with which the mice were inoculated contained 0.72 mg 17β-estradiol. The treatment of the mice was started on day 4 by either intraponeal injections of αIGF-IR scFv-Fc as in example 6 three times per week, by intraponeal injections of DOX (2 mg/kg bodyweight) once a week, or by a combination of both.

### EXAMPLE 8

Effect of αIGF-IR scFv-Fc in Combination With Anti-estrogen Agent Tamoxifen (TAM) on Tumor Growth in Vivo.

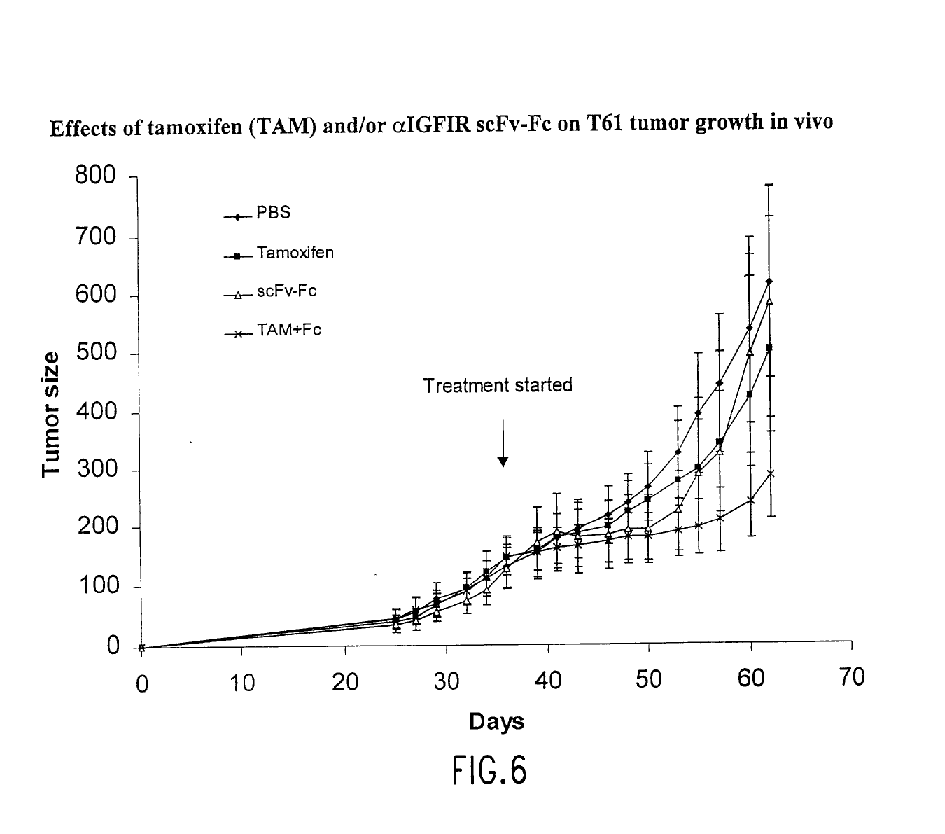

In combination with the anti-estrogen drug Tamoxifen (TAM), αIGF-IR scFv-Fc inhibits T61 tumor cell growth in vivo as is shown in FIG. 6. The observed inhibition of a combination treatment in these T61 tumor cells shows a synergistic effect. Female athymic mice were inoculated with T61 tumor cells. The treatment of the mice was started on day 36 by either intraponeal injections of αIGF-IR scFv-Fc as in example 6 three times per week, by subcutaneous implantation of 5 mg of a TAM pellet, or a combination of both.

What is claimed is:

1. An IGF-IR antagonist comprising a recombinant single-chain antibody capable of effectively inhibiting IGF-IR-agonist interaction.

2. An IGF-IR antagonist according to claim 1, wherein, the antibody is a chimeric single-chain recombinant antibody.

3. An IGF-IR antagonist according to claim 2, wherein the antibody is a humanized single-chain recombinant antibody.

4. An IGF-IR single-chain recombinant antibody capable of effectively inhibiting IGF-IR-agonist interaction wherein the recombinant antibody consists essentially of a V_{L} domain antigen binding portion linked to a V_{H} domain antigen binding portion through a peptide linker.

5. A recombinant antibody according to claim 4, wherein said antigen binding portions of said V_{L} and V_{H} domains are murine antibody domains.

6. A recombinant antibody according to claim 4, wherein the antibody consists essentially of a V_{L} domain antigen binding portion linked through a peptide linker to a V_{H} domain antigen binding portion linked to an immunoglobulin constant domain.

7. A recombinant antibody according to claim 6, wherein said V_{L} and V_{H} domains are murine antibody domains.

8. A recombinant antibody according to claim 6, wherein said immunoglobulin constant domain comprises C_{H2} and C_{H3} regions of a constant domain.

9. A recombinant antibody according to claim 8, wherein said C_{H2} and C_{H3} regions are from a non-murine mammalian immunoglobulin domain.

10. A recombinant antibody according to claim 9, wherein said C_{H2} and C_{H3} regions are human immunoglobulin regions.


SEQUENCE LISTING (2 sequences)

SEQ ID NO: 1 - protein, 15 aa, Murine
EVKVVESGGGLVKPG

SEQ ID NO: 2 - protein, 15 aa, Murine
DIVMTQSHKFMSTSV